(19) United States
(12) Patent Application Publication
(10) Pub. No.: US 2026/0076682 A1
(43) Pub. Date: Mar. 19, 2026

(54) MEDICAL IMPLANT SYSTEM

(71) Applicant: AURIGEN MEDICAL LIMITED, Galway (IE)

(72) Inventors: Tony O'HALLORAN, Athenry, Co. Galway (IE); John THOMPSON, Balgriffin, Dublin (IE); John KELLY, Salthill, Galway (IE); Matt MORAN, Claregalway, Co. Galway (IE); Kevin DONAGHEY, An Spideal, Co. Galway (IE)

(73) Assignee: AURIGEN MEDICAL LIMITED, Galway (IE)

(21) Appl. No.: 19/158,619

(22) PCT Filed: Feb. 22, 2024
(86) PCT No.: PCT/EP2024/054586

§ 371 Date: Sep. 10, 2025

(30) Foreign Application Priority Data
Feb. 22, 2023 (EP) 23158082.0

(51) Int. Cl. A61B 17/12; A61B 17/00; A61B 18/00; A61B 18/14; A61B 18/16; A61M 39/06

(52) U.S. Cl. CPC A61B 17/12172; A61B 17/12031; A61B 17/12122; A61B 18/1492; A61B 18/16; A61B 2017/00022; A61B 2017/00867; A61B 2017/12095; A61B 2018/00577; A61B 2018/00875; A61M 39/06

(57) ABSTRACT

A medical implant system (1) comprises an access sheath (2) having a lumen (3), an implant system disposed within and axially adjustable relative to the lumen (3) of the access sheath (2) and comprising an implant (6) that is radially adjustable from a contracted delivery configuration to a radially expanded deployed configuration detachably coupled to an implant delivery shaft (4) having a lumen (5), an anchoring system mountable within and axially adjustable relative to the lumen (5) of the implant delivery shaft (4) comprising an anchor (8) that is radially adjustable from a contracted delivery configuration to a radially expanded implant anchoring configuration detachably coupled to an anchor delivery shaft (7), and a handle (9) configured to be operatively coupled to the implant delivery shaft (4) and access sheath (2) and comprising an axial actuator to adjust the axial position of the implant delivery shaft (4) relative to the access sheath (2) to deploy and/or recapture the implant (6). The axial actuator comprises an external actuator sleeve (23) rotatably mounted to the main body (20) of the handle (9) for rotation about a longitudinal axis (21) of the handle (9). Methods of using the medical implant system (1) are also described.

### FIELD OF THE INVENTION

The present invention relates to a medical implant system. The invention also relates to a method of implanting and anchoring a medical implant is a body lumen such as a left atrial appendage of the heart.

### Background to the Invention

Systems for occlusion of a body lumen are described in the literature, for example US2020/0121324, US2020/0107836, WO2020/074738, WO2022/079235, and WO2022/129257. These systems generally include a nitinol cage implant that is radially adjustable from a contracted delivery configuration to a deployed radially expanded configuration, an implant delivery catheter detachably attached to the implant, an access catheter having a lumen for receipt of the delivery catheter to deliver the implant to a target body lumen, and a handle to which the access catheter and implant delivery catheter are coupled. The implant is usually detachable from the delivery catheter allowing the catheter to be withdrawn leaving the implant in-situ. The implant often comprises electrodes for tissue ablation, and electrical coupling systems for electrically coupling and decoupling the implant and delivery catheter. In addition, the prior art describes an anchoring system comprising an anchor that anchors the implant in a body lumen. Controlling all of the parts of such an occlusion system using a handle is challenging, as it requires separate axial and rotational movement of the delivery catheter and anchor delivery shaft. In addition, as deployment of the implant into contact tissue is a critical part of the process, controllable axial actuation of the implant is required.

It is an object of the invention to overcome at least one of the above-referenced problems.

### SUMMARY OF THE INVENTION

In a first aspect, the invention provides a medical implant system comprising:
an access sheath having a lumen;
an implant module mountable within and axially adjustable relative to the lumen of the access sheath and comprising an implant that is radially adjustable from a contracted delivery configuration to a radially expanded deployed configuration detachably coupled to an implant delivery shaft having a lumen;
an implant anchoring module comprising an anchor that is radially adjustable from a contracted delivery configuration to a radially expanded implant anchoring configuration; and
a handle operatively coupled to the implant delivery shaft and access sheath and comprising a main body and an axial actuator configured to adjust the axial position of the implant delivery sheath relative to the access sheath to deploy and/or recapture the implant.

The axial actuator suitably comprises an external actuator sleeve rotatably mounted to the main body of the handle for rotation about a longitudinal axis of the handle. This allows a surgeon to control the deployment and re-sheathing of the implant with great precision, with the diameter of the sleeve allowing minute adjustment of the axial position of the implant during deployment.

In any embodiment, the implant anchoring module is mountable within, and axially adjustable relative to, the lumen of the implant delivery shaft and comprises an anchor delivery shaft detachably coupled to the anchor. In another embodiment, the anchor is coupled to the implant for delivery therewith.

In any embodiment, the main body of the handle comprises a central bore and a central axis, and the axial actuator for the implant delivery shaft comprises:
an actuator arm disposed in the central bore configured for axial and non-rotational movement relative to the central bore; and
a translation mechanism configured to convert rotational movement of the axial actuator sleeve to axial movement of the actuator arm,
   wherein the implant delivery shaft is attached to the actuator arm for axial movement therewith.

In any embodiment, the translation mechanism comprises:
an axial slot formed in a sidewall of the main body;
a helical groove formed in an inner wall of the axial actuator; and
a radial pin coupled to the actuator arm that extends radially outwardly through the axial slot to engage the helical groove,
   whereby rotational movement of the axial actuator relative to the main body causes axial movement of the actuator arm and implant delivery shaft relative to the access sheath.

In any embodiment, the actuator arm comprises:
a distal section configured for axial movement along the central bore of the main body and comprising a central bore for receipt of the implant delivery shaft; and
a proximal section detachably coupled to the distal section,
   in which the implant delivery shaft is attached to the proximal section, whereby the proximal section can be decoupled from the distal section and rotated about the central axis to rotate the implant delivery shaft without rotation of the distal section of the actuator arm.

In any embodiment, the proximal section of the actuator arm is disposed proximally of the main body.

In any embodiment, the proximal section comprises a cylindrical hub with an internally threaded surface, and the proximal end of the distal section comprises an externally threaded fitting configured to engage the internally threaded surface of the cylindrical hub.

In any embodiment, the axial actuator sleeve is rotatably coupled to a distal end of the main body of the handle.

In any embodiment, a distal section of the main body has a waisted section, and the axial actuator sleeve is rotatably mounted to the waisted section.

In any embodiment, the anchor delivery shaft extends through the handle and has a proximal end comprising an anchor shaft hub that extends proud of a proximal end of the handle.

In any embodiment, the handle comprises a haemostasis valve for the anchor delivery shaft.

In any embodiment, the handle comprises a haemostasis valve for the access sheath. In any embodiment, the handle comprises a haemostasis valve for the implant delivery shaft.

In any embodiment, a working length of the implant delivery shaft and a working length of the access sheath are configured such that upon assembly of the implant module within the lumen of the access sheath and prior to deployment of the axial actuator of the handle, the implant is positioned no more than 100 mm, 75 mm, 50 mm, 30 mm, 20 mm, 10 mm, 8 mm or 5 mm proximally of a distal end of the access sheath. Thus, during assembly, when the implant delivery shaft is fully advanced distally along the access sheath, the implant will be disposed inside the access sheath adjacent the distal end of the sheath. Any further distal advancement of the implant relative to the access sheath requires actuation of the axial actuator of the handle.

In any embodiment, the distal end of the access sheath comprises a fluorescent or radio-opaque marker.

In any embodiment, the implant comprises a mesh cage with a proximal hub configured for detachable attachment to a distal end of the implant delivery sheath.

In any embodiment, the proximal hub of the implant comprises a fluorescent or radio-opaque marker.

In any embodiment, the anchor comprises a proximal anchoring hub and a plurality of anchoring arms extending distally from the distal anchoring hub.

In any embodiment, the anchoring arms are configured to self-deploy into contact with a wall off the body lumen when the anchoring arms are advanced distally beyond the proximal hub of the implant.

In any embodiment, the proximal anchoring hub is configured to abut the proximal hub of the implant when the anchoring arms are fully deployed to limit any further distal movement of the implant anchoring system.

In any embodiment, the proximal anchoring hub of the anchor is configured to nest within the proximal hub of the implant when the anchoring arms are fully deployed to limit any further distal movement of the implant anchoring system.

In any embodiment, the implant is radially self-adjustable from a contracted delivery configuration to a radially expanded medical implant configuration upon advancement of the implant distally of a distal end of the access sheath.

In any embodiment, the mesh cage comprises nitinol.

In any embodiment, the implant comprises one or more electrodes.

In any embodiment, a proximal end of the axial actuator comprises a plurality of graduated markings disposed along at least a part of an outer circumference of the axial actuator including a central marking, and the main body comprises a main body marking on an outer surface of the main body, wherein the markings are configured such that the central marking and main body marking align when the implant is disposed in the outer sheath just proximal of a distal end of the outer sheath.

In any embodiment, the access sheath comprises a dilator.

In any embodiment, the implant is configured to occlude a body lumen upon deployment of the body lumen.

In any embodiment, the implant is configured to occlude a blood vessel upon deployment in the blood vessel.

In any embodiment, the implant is configured to occlude a left atrial appendage of heart. In any embodiment, the implant comprises one or more sensors configured for sensing, for example, a parameter of tissue of a body lumen or a parameter of blood. The sensor may be disposed for example on a radially expansible part of the implant or may be disposed on or in the implant hub. Typically, the sensors are fixed to the implant.

In any embodiment, the anchoring system comprises one or more sensors configured for sensing, for example, a parameter of tissue of a body lumen or a parameter of blood. The sensors may be continuous with or attached to the medical implant anchor (e.g. attached to an anchoring arm or barb) or attached to the anchor delivery shaft.

In any embodiment, the implant or anchoring system may comprise a wireless communication module. The wireless communication module may be operatively coupled to the sensor and configured to relay sensing data to a receiver located on or external to the subject's body. The wireless communication module may be configured for inductive (contactless) electrical charging.

In any embodiment, the sensor is a light-addressable potentiometric (LAP) sensor. In any embodiment, the sensor has a self-cleaning element.

In any embodiment, the sensor is a micro electro-mechanical system (mems) sensor.

In any embodiment, the anchoring system comprises a tissue energising module, for example one or more electrodes. The treatment module may be continuous with or attached to the medical implant anchor (e.g. attached to an anchoring arm or barb) or attached to the anchor delivery shaft. The electrodes may be tissue ablation electrodes, such as non-thermal tissue ablation electrodes.

In any embodiment, the access sheath is steerable. The access sheath may be configured for bidirectional steering. The access sheath may comprise a fixed first curve and a steerable second curve. The access sheath may comprise one or more symmetrical curves. The access sheath may comprise one or more asymmetrical curves.

In any embodiment, the anchor (e.g. the proximal anchoring hub of the anchor or one or more anchoring arms of the anchor) comprises a sensor.

In any embodiment, the implant system is configured such that, in use, when the implant is deployed in the left atrial appendage and the proximal anchoring hub of the anchor abuts the proximal hub of the deployed implant, the sensor is configured to sense a parameter of the left atrium. Thus, the sensor may include a sensing probe or sensing surface that is disposed on or extends proximally away from a proximal surface of the proximal anchoring hub. The sensor may be configured to detect a parameter of the blood, for example pressure, for example left atrial pressure.

There is also described a medical implant system comprising:
an access sheath having a lumen;
an implant system disposed within and axially adjustable relative to the lumen of the access sheath and comprising an implant that is radially adjustable from a contracted delivery configuration to a radially expanded deployed configuration detachably coupled to an implant delivery shaft having a lumen;
an implant anchoring system mounted within and axially adjustable relative to the lumen of the implant delivery shaft comprising an anchor that is radially adjustable from a contracted delivery configuration to a radially expanded implant anchoring configuration detachably coupled to an anchor delivery shaft; and
a handle configured to be operatively coupled to the implant delivery shaft and access sheath and comprising an axial actuator to adjust the axial position of the
implant delivery shaft relative to the access sheath to deploy and/or recapture the implant,
   wherein the implant delivery shaft comprises an electrical supply lead configured to deliver electrical energy from an external energy source to the implant.

In any embodiment, the implant comprises a mesh cage comprising, or formed from, an electrically conducting material that is electrically coupled to the electrical supply lead of the implant delivery shaft.

In any embodiment, the implant comprises a mesh cage comprising tissue-contacting electrodes electrically coupled to the electrical supply lead of the implant delivery shaft.

In another aspect, the invention provides a method of implanting a medical implant, comprising the steps of:
advancing an access sheath comprising a lumen percutaneously until a distal end of the access sheath is positioned at a target location;
coupling to a handle an implant delivery shaft of an implant system comprising a medical implant detachably attached to the implant delivery shaft;
advancing the implant system through the lumen of the access sheath until the medical implant is positioned just proximally of a distal end of the access sheath;
actuating an axial retractor of the handle to advance the implant system relative to the access sheath to deploy the medical implant proud of the distal end of the access sheath, whereby the medical implant self-expands into a deployed configuration at the target location;
actuating a rotary actuator of the handle to rotate the implant delivery shaft to decouple the medical implant from the implant delivery shaft; and
percutaneously withdrawing the implant delivery shaft and the access sheath leaving the medical implant at the target location.

The method generally comprises anchoring the implant. An anchor may be part (or contiguous with) the implant (and delivered therewith) or may be separate to the implant and delivered separately.

Thus, in one embodiment, the method comprises the steps of:
advancing an access sheath comprising a lumen percutaneously until a distal end of the access sheath is positioned at a target location;
coupling to a handle an implant delivery shaft of an implant system comprising a medical implant detachably attached to an implant delivery;
advancing an anchoring system comprising a medical implant anchor detachably attached to an anchor delivery shaft through the implant delivery shaft until the anchor is positioned proximally of a distal end of the implant delivery shaft;
advancing the implant system through the lumen of the access sheath until the medical implant is positioned just proximally of a distal end of the access sheath;
actuating an axial retractor of the handle to advance the implant system relative to the access sheath to deploy the medical implant proud of the distal end of the access sheath, whereby the medical implant self-expands into a deployed configuration at the target location;
advancing the anchor delivery shaft along the implant delivery shaft to deploy the anchor at the target location whereby the deployed anchor anchors the medical implant at the target location;
rotating a proximal end of the anchor delivery shaft to decouple the anchor from the anchor delivery shaft;
actuating a rotary actuator of the handle to rotate the implant delivery shaft to decouple the medical implant from the implant delivery shaft; and
percutaneously withdrawing the implant delivery shaft, optionally the anchor delivery shaft, and the access sheath leaving the medical implant anchored at the target location.

In another embodiment, the method comprises the steps of:
advancing an access sheath comprising a lumen percutaneously until a distal end of the access sheath is positioned at a target location;
coupling to a handle an implant delivery shaft of an implant system comprising a medical implant detachably attached to an implant delivery, in which the medical implant comprises a deployable anchor;
advancing the implant system through the lumen of the access sheath until the medical implant is positioned just proximally of a distal end of the access sheath;
actuating an axial retractor of the handle to advance the implant system relative to the access sheath to deploy the medical implant proud of the distal end of the access sheath, whereby the medical implant and anchor self-expand into a deployed configuration at the target location;
actuating a rotary actuator of the handle to rotate the implant delivery shaft to decouple the medical implant from the implant delivery shaft; and
percutaneously withdrawing the implant delivery shaft and the access sheath leaving the medical implant anchored by the anchor at the target location.

In any embodiment, the axial retractor of the handle comprises an external actuator sleeve rotatably mounted to the main body of the handle about a longitudinal axis of the handle, wherein the step of actuation of the axial retractor comprises rotating the external actuator handle relative to the main body of the handle.

In any embodiment, the medical implant comprises a mesh cage with a proximal hub having a proximal hub lumen, and the anchor comprises a proximal anchoring hub and a plurality of anchoring arms extending distally from the proximal anchoring hub.

In any embodiment, the anchoring system is pre-loaded such that the proximal anchoring hub is disposed proximally of the proximal hub of the mesh cage and the anchoring arms are disposed in the implant delivery shaft.

In any embodiment, the step of advancing the anchoring system comprises advancing the anchor until the proximal anchoring hub abuts the proximal hub lumen.

In any embodiment, the anchor delivery shaft extends through a central lumen of the handle and comprises a proximal anchor delivery shaft section disposed proximally of the handle for manual adjustment of the anchor system.

In any embodiment, the main body of the handle comprises a central bore, and the axial actuator for the implant delivery shaft comprises:
an actuator arm disposed in the central bore configured for axial and non-rotational movement relative to the central bore; and
a translation mechanism configured to convert rotational movement of the external actuator sleeve to axial movement of the actuator arm,
wherein the implant delivery shaft is attached to the actuator arm for axial movement therewith.

In any embodiment, the actuator arm comprises a shaft section detachably coupled to the rotary actuator, in which the implant delivery shaft is attached to the rotary actuator, wherein the step of actuating the rotary actuator comprises decoupling the rotary actuator from the shaft section of the actuator arm, and rotating the rotary actuator to rotate the implant delivery shaft relative to the main body of the handle to decouple the implant delivery shaft from the anchored implant.

In any embodiment, the method comprises a step of advancing a dilator through the lumen of the access sheath, wherein the step of advancing the access sheath comprises advancing the access sheath and dilator.

In any embodiment, the method comprises a step of retracting the dilator from the access sheath prior to advancing the implant system through the lumen of the access sheath.

In any embodiment, the access sheath comprises a first haemostasis valve disposed at a proximal end thereof, wherein the step of advancing the implant system through the lumen of the access sheath comprises opening the haemostasis valve and inserting the implant system into the access sheath through the first haemostasis valve.

In any embodiment, the step of advancing the implant system through the lumen of the access sheath comprises coupling a loading tool comprising a second haemostasis valve to a distal end of the implant delivery shaft, coupling the second haemostasis valve to the first haemostasis valve, opening the second haemostasis valve, and advancing the implant system through the first and second haemostasis valves and along the access sheath.

In any embodiment, the method comprises attaching the loading tool to a distal end of the handle.

In any embodiment, the implant comprises a treatment or sensing module, in which the method comprises a step of treating or sensing prior to or after the implant has been anchored and prior to decoupling the implant delivery shaft from the implant.

In any embodiment, the implant comprises a treatment module, for example a tissue energising module comprising, for example, one or more electrodes, in which the treatment step comprises actuating the tissue energising module to for example treat the tissue at the target location. In one embodiment, the method comprises actuating one or more electrode of the tissue energising module to ablate tissue at the target location.

In any embodiment, the or each electrode of the implant is electrically coupled to an electrical controller via an electrically conducting wire, in which the method comprises actuating the electrical controller to energise the or each electrode.

In any embodiment, the method comprises the steps of:
actuating the axial actuator of the handle to at least partially re-sheath the medical implant prior to deployment of the anchor;
adjusting the axial position of the medical implant relative to the access sheath; and
actuating the axial actuator of the handle to re-deploy the medical implant.

In any embodiment, the method comprises the steps of:
after the anchor has been deployed, retracting the anchor delivery shaft until the anchoring arms are detached from tissue of the target location and optionally re-sheathed in the implant delivery shaft;
adjusting the rotational and/or axial position of the anchor; and
advancing the anchor delivery shaft until the anchoring arms are engaged with tissue of the target location.

In any embodiment, the steps of positioning the access sheath, deploying the implant, or deploying the anchor are performed under imaging, for example X-ray or fluoro-contrast imaging.

In any embodiment, the method is a method of occluding a body lumen, in which the medical implant is configured to occlude a body lumen when deployed in the body lumen.

In any embodiment, the body lumen is a blood vessel.

In any embodiment, the body lumen is a left atrial appendage of the heart.

In another aspect, the invention provides a system to ablate tissue of a body lumen, the system comprising:
a tissue ablation module comprising a radially expansible body configured for radial expansion from a radially contracted delivery configuration to a radially expanded tissue ablation configuration and at least one tissue ablation electrode disposed an external body lumen facing surface of the radially expansible body;
an elongated catheter having electrical conducting elements electrically coupled to the electrodes; and
a generator configured for electrical coupling to a proximal end of the catheter to deliver pulse field ablation energy comprising at least one pulse train of energy to the electrodes via the electrical conducting elements of the catheter.

In any embodiment the at least one pulse train of energy comprises or consists essentially of asymmetrical pulses.

In any embodiment the at least one pulse train of energy comprises or consists essentially of biphasic pulses.

In any embodiment the at least one pulse train of energy comprises or consists essentially of symmetrical pulses.

In any embodiment the at least one pulse train of energy comprises or consists essentially of monophasic pulses.

In any embodiment the at least one pulse train of energy comprises or consists essentially of asymmetrical biphasic pulses.

The invention also provides a method of ablating tissue of a body lumen that employs a system of the invention, comprising the steps of:
advancing the catheter and radially expansible body transluminally to the body lumen;
deploying the radially expansible body in the body lumen such that the energy delivery surface of at least some of the electrodes contact tissue of the body lumen; and
delivering by the generator pulse field ablation energy comprising at least one pulse train of energy to the electrodes via the electrical conducting elements of the catheter.

In any embodiment, the at least one pulse train of energy comprises or consists essentially of asymmetrical biphasic pulses, asymmetrical monophasic pulses, symmetrical biphasic pulses or symmetrical monophasic pulses.

In any embodiment, the generator delivers, or is configured to deliver, at least one pulse train of energy (typically at a predetermined frequency) comprising pulses having a positive voltage of 500 V to 2500 V.

In any embodiment, the generator delivers, or is configured to deliver, at least one pulse train of energy (typically at a predetermined frequency) comprising pulses having a negative voltage of 500 V to 2500 V.

In any embodiment, the generator delivers, or is configured to deliver, at least one pulse train of energy (typically at a predetermined frequency) comprising pulses having a positive pulse width of 2 μs to 20 μs.

In any embodiment, the generator delivers, or is configured to deliver, at least one pulse train of energy (typically at a predetermined frequency) comprising pulses having a negative pulse width of 2 μs to 20 μs.

In any embodiment, the generator delivers, or is configured to deliver, at least one pulse train of energy (typically at a predetermined frequency) comprising pulses having a switch time of 2 μs to 200 μs.

In any embodiment, the generator delivers, or is configured to deliver, at least one pulse train of energy (typically at a predetermined frequency) comprising pulses having a negative pulse delay of 2 μs to 20 μs.

In any embodiment, the generator delivers, or is configured to deliver, at least one pulse train of energy (typically at a predetermined frequency) comprising pulses comprising 5 to 100 pulses per burst.

In any embodiment, the generator delivers, or is configured to deliver, at least one pulse train of energy (typically at a predetermined frequency) comprising pulses having a burst delay of 1 ms to 1000 ms.

In any embodiment, the generator delivers, or is configured to deliver, to deliver 1 to 100 pulse trains of energy.

In another aspect, the invention provides a medical implant system comprising:
an access sheath having a lumen;
an implant module mountable within and axially adjustable relative to the lumen of the access sheath and comprising an implant that is radially adjustable from a contracted delivery configuration to a radially expanded deployed configuration detachably coupled to an implant delivery shaft having a lumen; and
an implant anchoring module comprising an anchor and an anchor delivery shaft detachably coupled to the anchor, wherein the anchor is radially adjustable from a contracted delivery configuration to a radially expanded implant anchoring configuration,
   wherein the implant anchoring module is mountable within, and axially adjustable relative to, the lumen of the implant delivery shaft.

In any embodiment, the anchor comprises a proximal anchoring hub and a plurality of anchoring arms extending distally from the distal anchoring hub.

In any embodiment, the anchoring arms are configured to self-deploy into contact with a wall off the body lumen when the anchoring arms are advanced distally beyond the proximal hub of the implant.

In any embodiment, a proximal face of the implant comprises a recessed conduit and the proximal anchoring hub is configured to nest within the recessed conduit when the anchoring arms are fully deployed to limit any further distal movement of the implant anchoring system.

In any embodiment, the proximal face of the implant has an annular recessed section surrounding the recessed conduit, wherein a distal end of the conduit is disposed distally (for example by 1-2 mm) of the annular recessed section of the proximal face of the implant. Thus, when the anchoring arms are advanced through the recessed conduit and deploy radially outwardly, they distal end of the conduit prevents the arms coming into contact with the annular recessed section of the implant when they pivot radially outwardly into a deployed position.

In any embodiment, the system comprises a handle operatively coupled to the implant delivery shaft and access sheath and comprising a main body and an axial actuator configured to adjust the axial position of the implant delivery sheath relative to the access sheath to deploy and/or recapture the implant.

In another aspect, the invention provides a method of implanting a medical implant, comprising the steps of:
advancing an access sheath comprising a lumen percutaneously until a distal end of the access sheath is positioned at a target location;
coupling to a handle an implant delivery shaft of an implant system comprising a medical implant detachably attached to the implant delivery shaft;
advancing the implant system through the lumen of the access sheath until the medical implant is positioned just proximally of a distal end of the access sheath;
advancing (optionally by means of actuation of the handle) the implant system relative to the access sheath to deploy the medical implant proud of the distal end of the access sheath, whereby the medical implant self-expands into a deployed configuration at the target location;
advancing an anchor module comprising an anchor detachably coupled to a distal end of an anchor delivery shaft along the implant delivery shaft to deploy the anchor at the target location whereby the deployed anchor anchors the medical implant at the target location;
decoupling the anchor from the anchor delivery shaft and the medical implant from the implant delivery shaft; and
percutaneously withdrawing the implant delivery shaft, anchor delivery shaft (and optionally the access sheath) leaving the medical implant at the target location. In any embodiment, the implant delivery shaft comprises a lumen configured to receive the anchor module. In any embodiment, the method comprises advancing the anchor and anchor delivery shaft through the lumen of the implant delivery shaft.

In any embodiment the handle comprises a first actuator to rotate the implant delivery shaft and a second actuator to rotate the anchor delivery shaft.

Other aspects and preferred embodiments of the invention are defined and described in the other claims set out below.

### BRIEF DESCRIPTION OF THE FIGURES

FIG. 1 is a side elevational view of a medical implant system according to the invention prior to advancement of the access sheath through the access sheath haemostasis valve.

FIG. 2 Is a Sectional View Taken Along the Lines A-A of FIG. 1.

FIG. 3 is detailed view of part of the medical implant system of FIG. 2 showing the distal end of the access sheath with the undeployed implant sheathed within the access sheath and the undeployed anchor sheathed within the implant delivery shaft.

FIG. 4 is a side elevational view of a medical implant system of FIGS. 1 to 3 shown after advancement of the access sheath through the access sheath haemostasis valve, after deployment of the implant and prior to deployment of the anchor.

FIG. 5 is a sectional view taken along the lines G-G of FIG. 4.

FIG. 6 is detailed view of part of the medical implant system of FIG. 5 showing the distal end of the access sheath with the implant deployed distally of the access sheath and the undeployed anchor sheathed within the implant delivery shaft.

FIG. 7 is a side elevational view of a medical implant system of FIGS. 1 to 3 after deployment of the implant and during deployment of the anchor, where the anchoring arms have not yet self-deployed into the splayed tissue-anchoring position.

FIG. 8 is a sectional view taken along the lines L-L of FIG. 7.

FIG. 9 is detailed view of part of the medical implant system of FIG. 8 showing the distal end of the access sheath with the implant deployed distally of the access sheath and the anchor advanced into a partially deployment position, and the proximal anchoring hub nested within the proximal hub of the implant.

FIG. 10 is a side elevational view of a medical implant system of FIGS. 1 to 3 shown after full deployment of the anchoring arms into an outwardly splayed, tissue anchoring, position.

FIG. 11 is a sectional view taken along the lines N-N of FIG. 10.

FIG. 12 is detailed view of part of the medical implant system of FIG. 11 showing the anchoring arms splayed outwardly into an implant anchoring position and the anchor delivery shaft detached from the proximal anchoring hub.

FIG. 13 is a side elevational view of a medical implant system of FIGS. 1 to 3 during uncoupling of the implant delivery shaft from the implant, where one of the surgeon's hands is holding the proximal section of the actuator arm after it has been uncoupled from the distal section of the actuator arm.

FIG. 14 is a sectional view taken along the lines N-N of FIG. 10.

FIG. 15 is detailed view of part of the medical implant system of FIG. 11 showing the anchor delivery shaft re-sheathed inside the lumen of the implant delivery sheath, and the implant delivery shaft rotatably detached from the implant by rotating the proximal section of the actuator arm (rotary actuator) after it has been uncoupled from the distal section of the actuator arm.

FIGS. 16A to 16E are schematic illustrations of the use of the system of the invention in which:

FIG. 16A illustrates a distal end of any access sheath positions at an ostium of a left atrial appendage (LAA), with an implant (in a stowed delivery configuration) and implant delivery shaft disposed in an access sheath with the undeployed implant disposed just proximal of the distal end of the access sheath and the anchor and anchor delivery shaft disposed in the implant delivery sheath with the distal tips of the anchoring arms disposed just distal of the proximal hub of the implant;

FIG. 16B illustrates deployment of the implant by advancement of the implant delivery shaft proximally by about 50 mm until the implant is exposed distally of the access sheath whereupon it deploys radially to circumferentially engage the ostium of the LAA;

FIG. 16C illustrates deployment of the anchor by advancement of the anchor delivery shaft distally by about 20 mm until the proximal anchor hub abuts and nest within the proximal hub of the implant and the anchoring arm disposed within the implant and splayed outwardly into engagement with the tissue through apertures in the wall of the implant to anchor the implant in position;

FIG. 16D illustrates the anchor delivery shaft uncoupled from the proximal hub of the anchor and re-sheathed inside the implant delivery shaft; and

FIG. 16E illustrates the implant delivery shaft uncoupled from the proximal hub of the implant and re-sheathed inside the implant delivery shaft and re-sheathed within the access sheath. Once this is completed, the access sheath containing the implant delivery shaft and anchor delivery shaft is retracted percutaneously leaving the implant anchored in the LAA.

FIG. 17 illustrates an anchor system comprising an anchor delivery shaft attached to a proximal hub of an anchor.

FIG. 18 illustrates an anchor comprising eight anchoring arms (shown in a splayed tissue engaging position) attached to a proximal hub.

FIG. 19 illustrates a medical implant with a proximal hub and a distal end of an implant delivery sheath prior to coupling to the proximal hub.

FIG. 20 illustrates the proximal end of the handle showing the decoupling of the rotary actuator from the proximal end of the actuator arm shaft. Once uncoupled, the rotary actuator may be rotated to rotate the implant delivery shaft to uncouple the implant delivery shaft from the implant. Before uncoupling, rotation of the implant delivery shaft is not possible as it is attached to the actuator arm.

FIG. 21 is an image of the recessed hub taken from inside the mesh cage implant showing the distal tips of the anchoring arms projecting through the proximal hub of the mesh cage prior to deployment of the anchor. This is the position of the anchoring arms in the pre-loaded configuration and prior to deployment of the anchor.

FIG. 22 illustrates the assembly of access sheath and dilator and first haemostasis valve.

FIG. 23 illustrates a proximal end of the haemostasis valve clip arm showing a proximal end of the dilator.

FIG. 24 illustrates access sheath and dilator being advanced percutaneously into the femoral vein over a guidewire. The proximal end of the dilator is not fully abutted against the haemostasis valve.

FIG. 25 illustrates the access sheath and dilator tracked over the guidewire along the femoral artery and into the left atrium.

FIG. 26 illustrates the pre-assembly of the handle, implant system (implant and implant delivery shaft) and second haemostasis valve.

FIG. 27 illustrates the anchor system (anchor plus anchor delivery shaft) loaded in the handle after being advanced through the third haemostasis valve and implant delivery sheath to a position where the anchor is located just proximal of the proximal hub of the implant.

FIG. 28 illustrates the second haemostasis valve being attached to the first haemostasis valve while the access sheath is position in the vasculature. At this stage, the dilator has been retracted and removed from the access sheath.

FIG. 29 illustrates the second haemostasis valve being turned anti-clockwise to allow advancement of the implant system and anchor system through the second haemostasis valve and along the sheath.

FIG. 30 illustrates the second haemostasis valve coupled to the handle after the implant system and anchor system have been advanced through the access sheath to a position where the implant is positioned just proximal of a distal end of the access sheath.

FIG. 31 illustrates the implant positioned just proximal of a distal end of the access sheath.

FIG. 32 illustrates the handle and implant system (and anchor system) advanced through the access sheath.

FIG. 33 illustrates the use of the rotary sleeve to deploy the implant from the distal end of the access sheath.

FIG. 34A is a side view of an implant of the invention.

FIG. 34B is a perspective exploded view of the implant showing the components of the implant including a cover, circumferential array of electrodes, and mesh cage.

FIG. 34C is a side view of the implant of FIG. 34A with the front part of the cover cut-away for clarity.

FIG. 35A is a sectional side view of a system of the invention comprising an implant and anchor in a deployed configuration with the proximal anchor hub nesting in the recessed conduit (hub) of the implant.

FIG. 35B is a detailed view of the recessed conduit of the implant with the anchor module removed an illustrating the distal end of the recessed conduit extending distally of the annular recessed section of the proximal face of the implant.

FIG. 35C is a detailed view of the recessed conduit of the implant with the proximal anchor hub nesting in the recessed conduit and the anchor arms deployed.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,”and “at least one”are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the increase in levels of a tight junction protein). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

In the context of treatment as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

“Implant” or “medical implant” means an apparatus configured for implantation in a subject's body, especially a body lumen, especially implantation in the heart for example partially or fully within the left atrial appendage. The implant may be for occlusion of a body lumen, for treatment or sensing, or both. When the implant is an occlusion apparatus, it is configured for actuation/deployment to at least partially or fully fluidically occlude a body lumen. The implant is typically detachably connected to an implant delivery shaft/catheter which delivers the implant to the target site, and typically remains attached during occlusion, sensing and/or energy delivery treatments and in one embodiment is generally detached after the energy delivery treatment and removed from the body leaving the occlusion apparatus implanted in the body lumen. The implant generally includes a central proximal connection hub (also referred to herein as a “proximal anchor hub” and “recessed conduit”) for attaching to the delivery catheter, and a radially expansible body. Occlusion may be complete occlusion (closing) of the body lumen or partial occlusion (narrowing of the body lumen or near complete occlusion). The implant typically comprises a body that is expansible from a contracted delivery configuration to an expanded deployed configuration. The body may take many forms, for example a wireframe structure formed from a braided or meshed material (e.g. a mesh cage). Examples of expandable wireframe structures suitable for transluminal delivery are known in the literature and described in, for example, WO01/87168, U.S. Pat. No. 6,652,548, US2004/219028, U.S. Pat. Nos. 6,454,775, 4,909,789, 5,573,530, WO2013/109756. Other forms of bodies suitable for use with the present invention include plate or saucer shaped scaffolds, or stents. In one embodiment, the body is formed from a metal, for example a shape-memory metal such as nitinol. The body may have any shape suitable for the purpose of the invention, for example cylindrical, discoid or spheroid. In one preferred embodiment, the apparatus comprises a cylindrical body, for example a cylindrical cage body. In one embodiment, the body comprises a tissue energising module. In one embodiment, the ablation device comprises an array of electrodes, typically a circumferential array. In one embodiment, the array of electrodes is configured to deliver pulsed field ablation to the tissue. In one embodiment, a distal face of the radially expansible body comprises a covering configured to promote epithelial cell proliferation. In any embodiment, the electrodes are coupled to the mesh cage. In any embodiment, the electrodes are coupled to the covering. In any embodiment, the electrodes are coupled to the mesh cage and at least part of each electrode is covered by the covering. In one embodiment, the body comprises a stepped radial force stiffness profile from distal to proximal device. In one embodiment, the body comprises a metal mesh cage scaffold. In one embodiment, a coupling (e.g. the connecting hub) between the body and the catheter member is located distally to the left atrial facing side of the body. In one embodiment, the body in a deployed configuration has a radial diameter at least 10% greater than the radial diameter of the left atrial appendage at a point of deployment. In one embodiment, the furthermost distal part is configured to be atraumatic to cardiac tissue. In one embodiment, the body comprises a braided mesh scaffold that in one embodiment is conducive to collagen infiltration on thermal energy delivery to promote increased anti migration resistance.

“Body lumen” means a cavity in the body, and may be an elongated cavity such as a vessel (i.e. an artery, vein, lymph vessel, urethra, ureter, sinus, auditory canal, nasal cavity, bronchus) or an annular space in the heart such as the left atrial appendage, left ventricular outflow tract, the aortic valve, the mitral valve, mitral valve continuity, or heart valve or valve opening.

“Transluminal delivery” means delivery of the implant to a target site (for example the heart) heart through a body lumen, for example delivery through an artery or vein. In one embodiment, the device of the invention is advanced through an artery or vein to deliver the occlusion apparatus to the left atrium of the heart and at least partially in the LAA. In one embodiment, the device is delivered such that the distal part is disposed within the LAA, and the proximal part is disposed in the left atrium just outside the LAA. In one embodiment, the device is delivered such that the distal part is disposed within the LAA, and the proximal part is disposed in the left atrium abutting a mouth of the LAA. In one embodiment, the device is delivered such that both the distal and proximal parts are disposed within the LAA.

“Anchor” means a device that can be adjusted between a delivery configuration for percutaneous delivery to a target location and a deployed configuration where the device anchors the implant at the target location, for example a wall of a body lumen. In one embodiment the anchor comprises an anchoring arm, and preferably an array of anchoring arms that can be deployed to anchor the implant in the body lumen. The anchoring arm(s) may be made from a shape memory material, such as nitinol. The anchoring arm(s) are generally adjustable (e.g. pivotally adjustable about their proximal end) from an axial position prior to deployment (in which the anchoring arms are generally bunched together along or close to a longitudinal axis of the device) to an outwardly splayed configuration. When fully deployed a distal end of at least some of the anchoring arms generally extend through apertures in the radially expansible body to engage tissue. The anchoring arms are generally biased into the outwardly splayed configuration and deployed by releasing a constraining member, such as a deployment catheter. This is also referred to herein as self-deployment. In some of the embodiments described herein, the anchoring arms are attached to the proximal hub of the implant. The anchoring module may comprise an anchoring module hub. The proximal connecting hub and hub of the anchoring module may be configured to abut to prevent further axial movement of the anchor distally relative to the implant. The anchoring module may include at least 2, 3, 4, 5 or 6 anchoring arms. In any embodiment, one or more of the anchoring arms comprise a tissue treatment element such as a tissue ablation electrode. The anchor arms, especially tips of the anchor arms, may comprise a radio-opaque material. This assists with placement and contact determination. The anchor may comprise a sensor, for example a sensor of a blood parameter (e.g. pressure), for example a left atrial pressure sensor.

“Cover”: Typically, the implant has a proximal cover which is impermeable to blood and that may include a re-closable aperture, for example an overlapping flap of material. The re-closable aperture may be configured to allow proximal movement of the implant delivery shaft or anchor delivery shaft through the aperture while preventing blood flow through the aperture. The implant may include a connecting hub distal of the cover, and configured for coupling with a distal end of the implant delivery shaft. The cover may be configured to act as a scaffold for in-vivo endothelialisation, and the cover may be configured to become impermeable to blood as a result of in-vivo epithelisation. The cover may be formed from a woven mesh material.

“Covering/cover configured to act as a scaffold for in-vivo endothelialisation” means a material that is use promotes epithelialisation of the distal or proximal body. In one embodiment, the covering is a membrane that comprises agents that promote epithelial cell proliferation. Examples include growth factors such as fibroblast growth factor, transforming growth factor, epidermal growth factor and platelet derived growth factor, cells such as endothelial cells or endothelial progenitor cells, and biological material such as tissue or tissue components. Examples of tissue components include endothelial tissue, extracellular matrix, sub-mucosa, dura mater, pericardium, endocardium, serosa, peritoneum, and basement membrane tissue. In one embodiment, the covering is porous. In one embodiment, the covering is a biocompatible scaffold formed from biological material. In one embodiment, the covering is a porous scaffold formed from a biological material such as collagen. In one embodiment, the covering is a lyophilised scaffold.

The implant or anchor employed in the system of the invention may include a tissue energising module. “Tissue energising module” as used herein refers to an array of tissue treating elements configured to treat tissue by application of, e.g., heat, cold, sound, light, microwave energy, or RF energy. The elements may be electrodes. The electrodes disposed on the implant may be configured for electrical coupling with an electrical controller. The electrodes are generally individually coupled with the controller to allow electrode specific energising of the electrode. They array of electrodes is generally arranged on the implant in a circumferential arrangement and configured to contact the wall of the body lumen in a circumferential pattern when the apparatus is deployed. The electrodes are typically configured to deliver energy, generally PFA, circumferentially around the wall of the body lumen. The electrodes may also function as sensors to detect an electrical parameter of the tissue of the wall of the body lumen, for example electrical impedance or electrical activity (voltage), or electrical mapping of the LAA or heart. The electrodes may be configured to measure an electrical parameter radially across the wall of the body lumen, or circumferentially along a section of the circumference of the wall of the body lumen. Generally, measuring an electrical parameter such as electrical impedance radially across the wall of the body lumen employs an electrode of the array of electrodes and an earth or ground pad placed on the patient's body, often the leg. Measuring an electrical parameter such as electrical impedance circumferentially along a section of the body lumen employs two electrodes where one electrode functions as an energising electrode and the other functions as a detecting electrode. The electrical parameter such as electrical impedance may be measured at one frequency or over a range of frequencies. The implant may comprise a battery, and the tissue energising module may be electrically coupled to the battery to power the electrodes. The implant may comprise a wireless communication module (which may be Bluetooth™ or RF based) to send and/or receive date to/from a remote source, for example an electrical controller, a computer or mobile communications device. The implant may comprise a power source configured for wireless inductance charging.

The system of the invention may be used to prevent or treat or diagnose a cardiac condition such as atrial fibrillation. The invention may also relate to a method of preventing or treating or diagnosing atrial fibrillation. “Atrial fibrillation” or “AF” is a common cardiac rhythm disorder affecting an estimated 6 million patients in the United States alone. AF is the second leading cause of stroke in the United States and may account for nearly one-third of strokes in the elderly. In greater than 90% of cases where a blood clot (thrombus) is found in the AF patient, the clot develops in the left atrial appendage (LAA) of the heart. The irregular heartbeat in AF causes blood to pool in the left atrial appendage, because clotting occurs when blood is stagnant, clots or thrombi may form in the LAA. These blood clots may dislodge from the left atrial appendage and may enter the cranial circulation causing a stroke, the coronary circulation causing a myocardial infarction, the peripheral circulation causing limb ischemia, as well as other vascular beds. The term includes all forms of atrial fibrillation, including paroxysmal (intermittent) AF and persistent and longstanding persistent AF (PLPAF).

The system of the invention may be used to prevent or treat or diagnose a cardiac condition such as an ischaemic event. The invention may also relate to a method of preventing or treating or diagnosing an ischaemic event. “Ischaemic event” refers to a restriction in blood supply to a body organ or tissue, resulting in a shortage of oxygen and glucose supply to the affected organ or tissue. The term includes stroke, a blockage of blood supply to a part of the brain caused by a blood clot blocking the blood supply to the brain and the resultant damage to the affected part of the brain, and transient ischaemic events (TIA's), also known as “mini-strokes”, which are similar to strokes but are transient in nature and generally do not cause lasting damage to the brain. When the restriction in blood supply occurs in the coronary arteries, the ischaemic event is known as a myocardial infarction (MI) or heart attack.

The implant and/or anchor may be self-deployable. The radially expansible body may be self-deployable. At least one of the anchoring arms may be self-deployable. The implant may comprise a shape memory material. At least one of the anchoring arms may be self-deployable.

### Exemplification

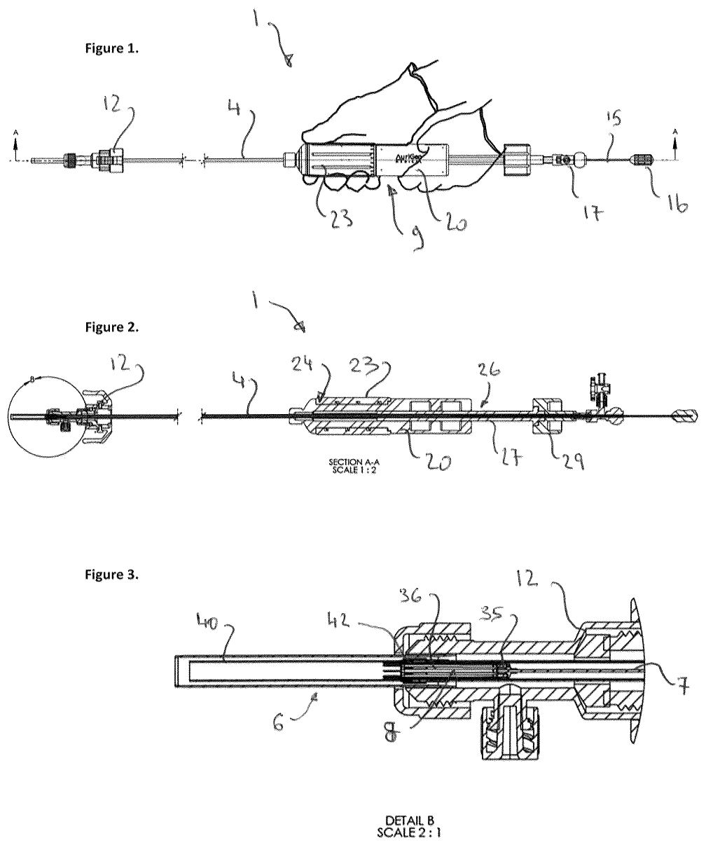

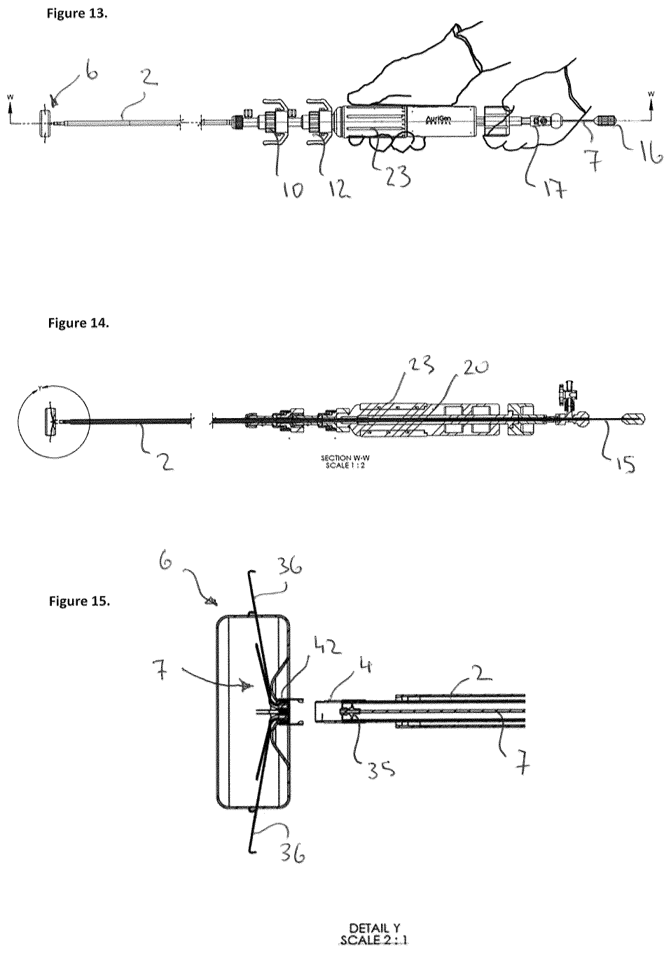

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention. Referring to the drawings and initially to FIGS. 1 to 15, there is illustrated a medical implant system according to the invention, indicated generally by the reference numeral 1, showing during assembly and use of the system to deliver and anchor a medical implant in the body lumen. The system 1 comprises an access sheath 2 with a lumen 3 extending along the full length of the sheath, an implant system comprising an implant delivery shaft 4 with a lumen 5 extending along the full length of the shaft, an implant 6 detachably counted to a distal end of the shaft 4, and an anchor system comprising an anchor delivery shaft 7 and anchor 8 detachably attached to a distal end of the anchor delivery shaft 7. The system also includes a handle 9, and a first haemostasis valve 10 for the implant delivery shaft and distal coupling conduit 11.

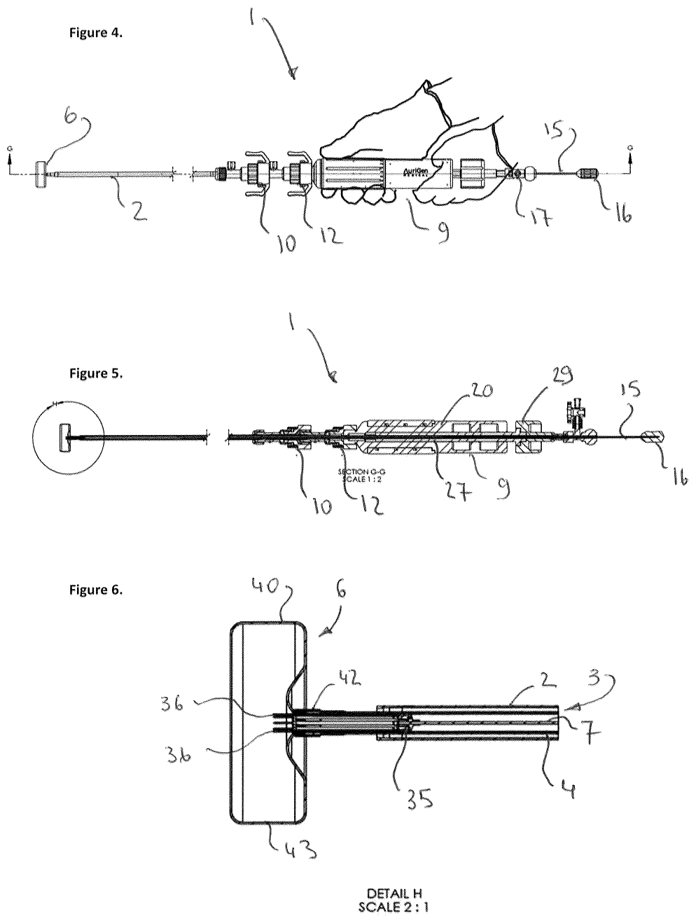

FIGS. 1 to 3 illustrate implant system, with the anchor system pre-loaded, prior to advancement through the access sheath 2. FIGS. 4 to 6 illustrate the implant delivery system fully advanced through the access sheath 2 and a second haemostasis valve 12 for the access sheath which is disposed distally of the first haemostasis valve 10. The first haemostasis valve 10 has been coupled to a distal end 14 of the handle 9 and the second haemostasis valve 12 has been coupled to the distal coupling conduit 11 of the first haemostasis valve 10. Referring to FIG. 6, the implant 6 is shown advanced distally of a distal end 13 of the access sheath 2 where it has radially self-deployed into a radially expanded configuration. At this point, most of the anchor 8 is disposed in the implant delivery shaft 4. The anchor delivery shaft 7 extends from the anchor 8 through the implant delivery shaft 4 and handle 9 providing a proximal section 15 with an anchor shaft hub 16. A third haemostasis valve 17 for the anchor delivery shaft 7 is attached to a distal end of the handle 9.

In more detail, the handle comprises a main body 20 having a longitudinal axis 21 and a central bore 22 formed along the axis 21, and an axial actuator comprising a sleeve 23 rotatably mounted to a recessed distal section 24 of the main body 20 for rotation about a longitudinal axis 21 of the handle 9. The axial actuator also comprises an actuator arm 26 having a shaft 27 with a central shaft bore 25 that is mounted within the central bore 22, and a translation mechanism configured to convert rotational movement of the sleeve 22 into axial movement of the actuator arm 26. The actuator arm 26 has a proximal section 28 comprising a rotary actuator 29 detachably coupled (e.g., threaded engagement) to a proximal end 30 of the shaft 27, and the distal end of the implant delivery sheath is attached to rotary actuator 29 via the third haemostasis valve 17.

Although not illustrated, the shaft 27 has a square cross section and the bore 22 of the main body has a complimentary cross section to allow axial movement of the shaft along the bore while preventing any rotational movement of the shaft in the bore. The rotary to axial movement translation mechanism comprises a slot (not shown) in the recessed distal section 24 of the main body, a helical groove 32 on an inside wall of the sleeve 22, and a follower pin (not shown) attached to the shaft 27 that extends through the slot and engages the helical groove 32. Thus, when the sleeve 22 is rotated about the axis 21 of the handle, this rotational movement is translated into axial movement of the shaft 27 (and actuator arm 26), resulting in axial movement of the implant delivery shaft 4 relative to the access sheath 2. The provision of an axial actuator for the deployment of the implant on the handle in the form of a rotatable sleeve that forms part of the external surface of the handle allows a surgeon to controllably deploy and re-sheath the implant with a great degree of precision as illustrated in FIGS. 1 to 4. In FIG. 1, the system is shown prior to deployment of the implant 6, with the actuator arm 26 located proximally relative main body of the handle, and in FIG. 4, after the implant has been deployed, the actuator arm is shown advanced distally through the main body of the handle. The axial position of the proximal section 28 of the handle (compare the position in FIGS. 1 and 4) provides a surgeon a visible cue as to the degree of deployment of the implant during the procedure.

Referring now to FIGS. 17 and 18, the anchor system comprises an elongated anchor shaft 7 with a proximal end 15 having a proximal anchor shaft hub 16, and anchor 8 comprising a proximal anchor hub 35 and eight anchoring arms 36 extending distally from the hub 35. A distal end of the anchor shaft 7 is externally threaded and the proximal anchor shaft hub 16 comprises corresponding internal threads to allow the shaft and anchor to be decoupled in-vivo by rotation of the anchor delivery shaft relative to the deployed anchor. In the figures, the arms are shown in their deployed, outwardly splayed, configuration. The anchor is formed with the arms biased into the deployed configuration so that they self-deploy when they are advanced beyond the constrains of the implant delivery sheath.

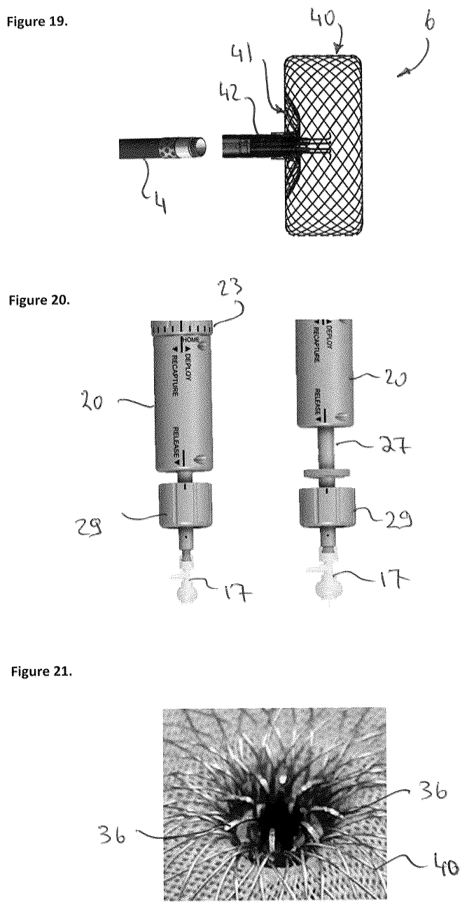

Referring now to FIG. 19, the implant 6 in this embodiment comprises a nitinol mesh cage 40 having a recessed proximal end 41 with a recessed hub 42, sidewall 43 and open proximal end. Although not shown, the proximal end of the mesh cage is covered by a blood impermeable cover so as to make the case blood impermeable. This is required when the implant is an occluding implant. An inside surface of the recessed hub 42 is threaded, and a distal end of the implant delivery shaft is externally threaded to allow the implant and implant delivery shaft to be decoupled in-vivo.

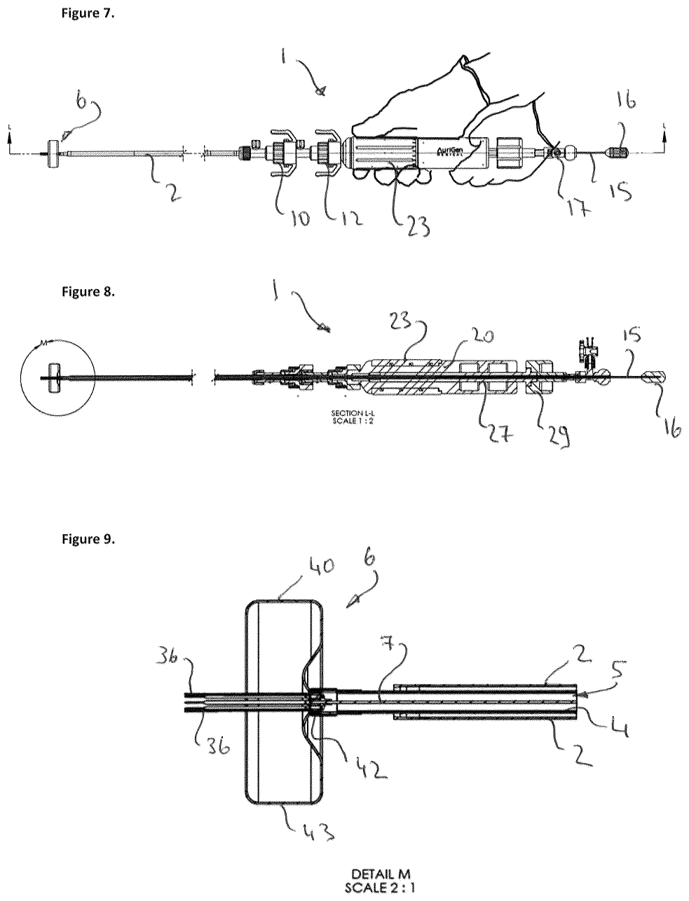

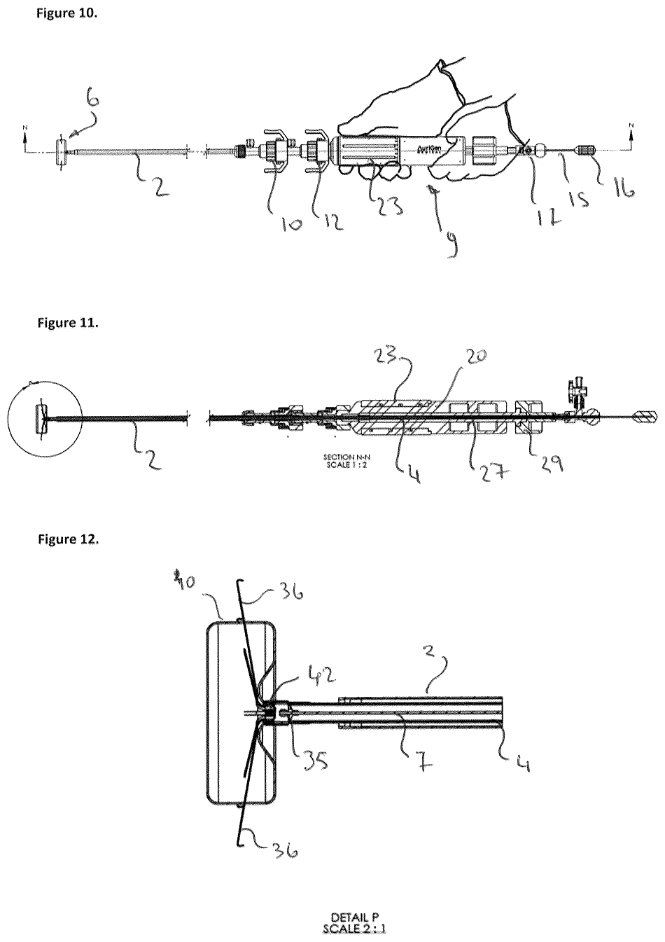

FIGS. 7 to 9 illustrate the system of the invention 1 during deployment of the anchor system. The anchor delivery shaft 7 has been advanced through the implant delivery shaft 4 from the position shown in FIG. 6 to a fully advanced position shown in FIG. 9. This is achieved by pushing the proximal anchor shaft hub 16 distally from the position shown in FIG. 4 to the position shown in FIG. 7. In this position, the proximal anchor hub 35 is nested within, and abuts, the recessed hub 41 of the implant, thus limiting any further distal movement of the anchor relative to the implant. Proximal movement of the anchor is limited by the anchoring arms engaging the tissue of the body lumen, and the configuration of the anchoring barbs at the tips of the anchoring arms which are swept proximally. The anchoring arms 36 are shown in their constrained delivery configuration but as soon as the arms are advanced through the proximal hub of the implant, they will self-deploy outwardly to engage the tissue of a body lumen through the gaps in the nesh cage 40 (illustrated in FIGS. 10 to 12).

Referring to FIG. 9, the system is shown after detachment of the anchor delivery shaft 7 from the proximal anchor hub 35. This is achieved by loosening the third haemostasis valve 17, gripping the proximal anchor shaft hub 16 and rotating the hub 16 anti-clockwise to unscrew the anchor delivery shaft from the deployed anchor, and then pulling the proximal anchor hub 35 proximally to re-sheath the anchor delivery shaft 7 within the implant delivery shaft 4.

Referring now to FIGS. 10 to 12, and FIG. 20, the system is shown during the decoupling of the implant 6 and implant delivery shaft 4. This is achieved by unscrewing the rotary actuator 29 from the proximal end 30 of the shaft 27 (thereby uncoupling the rotary actuator 29 from the axial actuator arm) and rotating the rotary actuator 29 in an anti-clockwise direction. As the implant delivery shaft 4 is attached to the rotary actuator 29 via the third haemostasis valve 17, this results in the rotation of the implant delivery shaft and decoupling of the implant delivery shaft from the anchored implant. Once uncoupled, the implant delivery shaft 4 is re-sheathed within the access sheath.

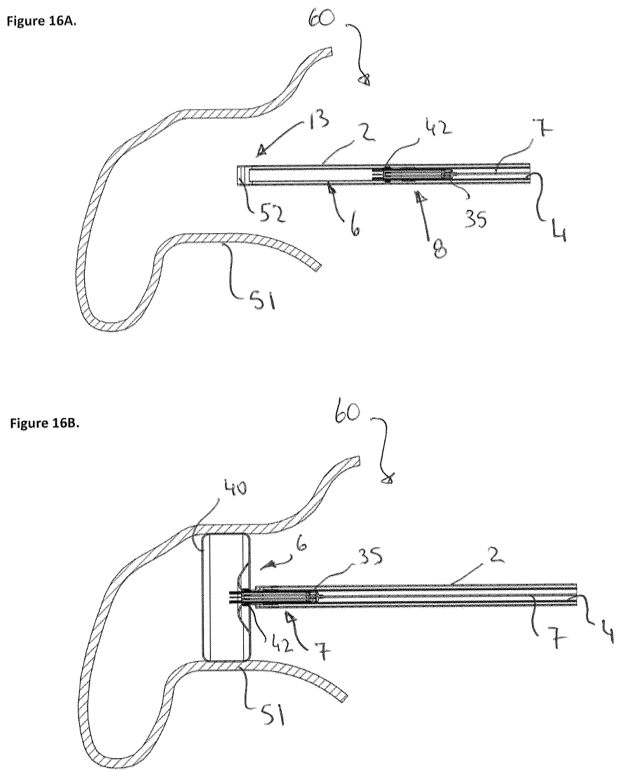

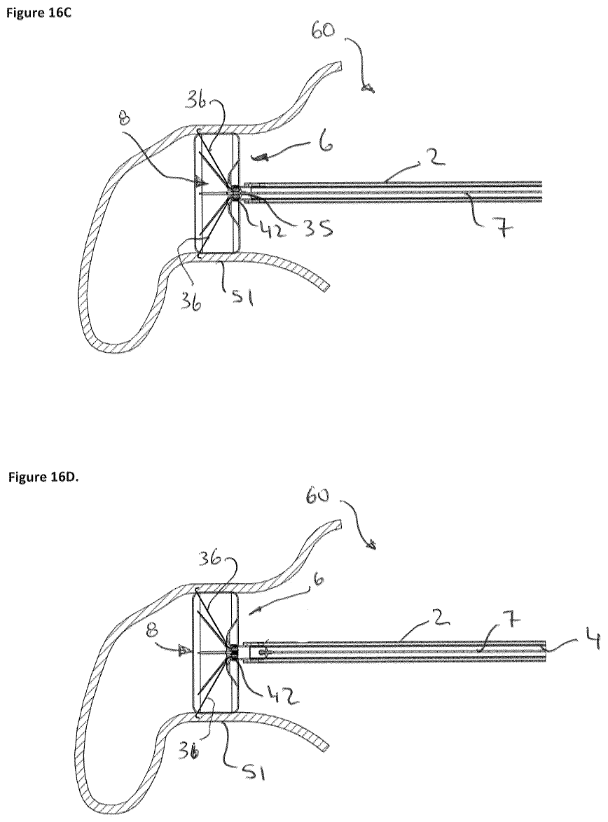

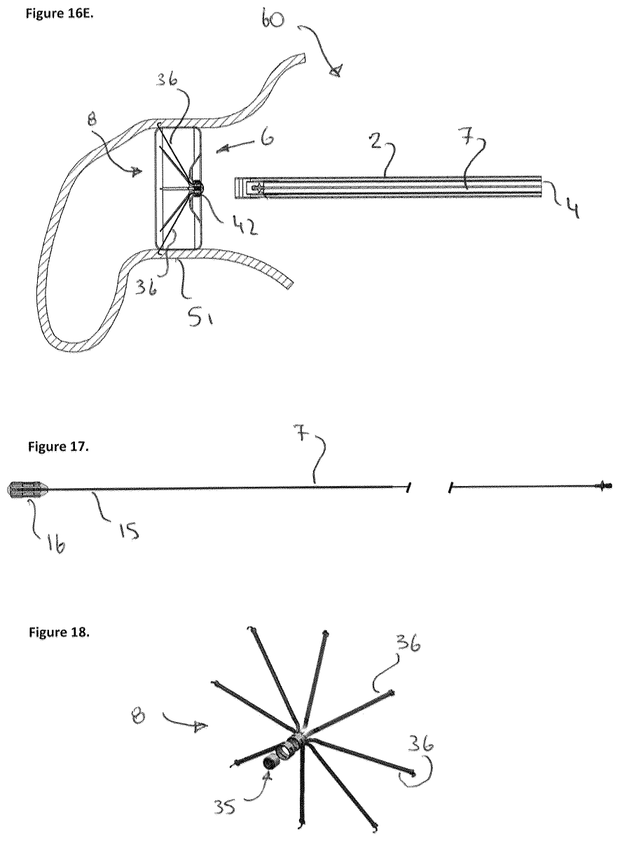

FIGS. 16A to 16E are schematic illustrations of the use of the system of the invention to implant an occluding implant 6 and anchor 8 in a left atrial appendage (LAA) 50 of a human heart, in particular showing how the implant and anchor are deployed in sequence prior to re-sheathing of the delivery shafts of the anchor and implant leaving the implant anchored in the LAA FIG. 16A illustrates the access sheath 2 positioned at an ostium 51 of a left atrial appendage (LAA), with an implant 6 (in a stowed delivery configuration) and implant delivery shaft 4 disposed in the access sheath 2 with the undeployed implant disposed just proximal of the distal end 13 of the access sheath 2 and the anchor 8 and anchor delivery shaft 7 disposed in the implant delivery sheath 4 with the distal tips of the anchoring arms disposed just distal of the proximal hub of the implant. The distal end 13 of the sheath 2 includes a radio-opaque marker band 52 which is visible under imaging and can be used to correctly position the sheath 2.

FIG. 16B illustrates deployment of the implant 6 by advancement of the implant delivery shaft 4 distally by about 20 mm until the implant is exposed distally of the access sheath whereupon it deploys radially to circumferentially engage the ostium of the LAA. As described previously, this is carefully performed by the surgeon by rotation of the actuator sleeve 23. The recessed hub 42 also includes a radio-opaque marker band (not shown) which is visible under imaging and can be used to correctly position the implant 6 relative to the sheath 2 and to ensure that the implant is fully deployed. At this stage, the anchor 8 is sheathed within the implant delivery shaft 4 with the distal tips of the anchoring arms 36 projecting through the proximal hub of the implant (as illustrated in FIG. 21).

FIG. 16C illustrates deployment of the anchor 8 by advancement of the anchor delivery shaft 7 distally (as described previously) by about 10 mm until the anchor proximal hub 35 abuts and nest within the proximal hub 42 of the implant 6 and the anchoring arms 36 disposed within the implant 6 and splayed outwardly into engagement with the wall 53 of the LAA through apertures in the wall of the implant to anchor the implant in position. At this stage, the implant is anchored to the wall 53 of the LAA and the implant is attached to the implant delivery catheter and the anchor is attached to anchor delivery catheter. The integrity of anchoring can be checked by pulling the proximal anchor shaft hub 16 proximally to ensure that the anchor does not move.

FIG. 16D illustrates the anchor delivery shaft 7 uncoupled from the proximal anchor hub 35. As described previously, this is performed by loosening the third haemostasis valve 17 and rotating the anchor delivery shaft 7 in an anti-clockwise direction until it is decoupled from the anchor 8. The anchor 8 is then re-sheathed inside the implant delivery shaft 4 by pulling the anchor delivery shaft 7 proximally using the proximal anchor hub 35. 4 FIG. 16E illustrates the implant delivery shaft uncoupled from the proximal hub 42 of the implant 6 and re-sheathed within the access sheath 2. Once this is completed, the access sheath can be retracted percutaneously.

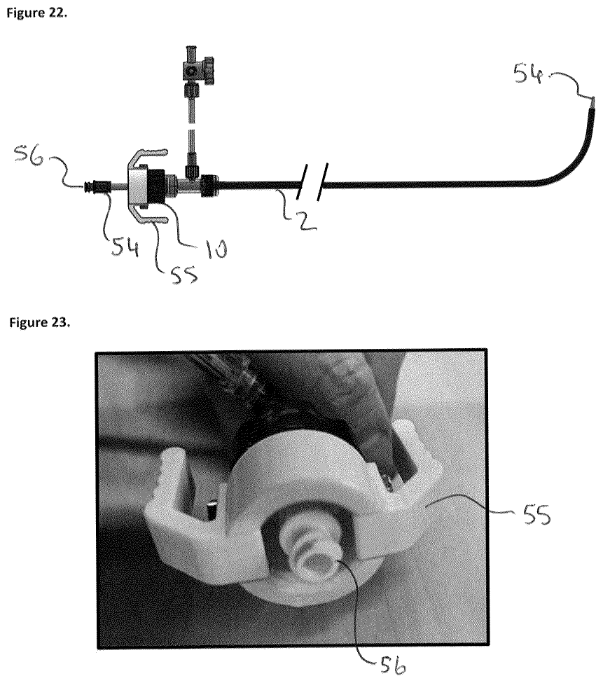

FIGS. 22 to 33 illustrate the assembly and use of a medical implant system of the invention (1).

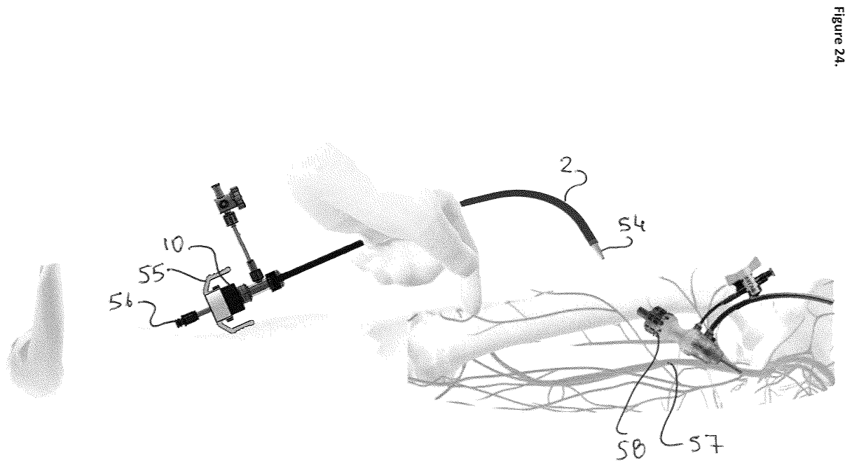

The first step (FIG. 22) comprises providing the access sheath 2 with the first haemostasis valve 10 attached to a proximal end thereof, and advancement of a dilator 54 through the lumen 3 of the access sheath 2. FIG. 23 illustrates a proximal end of the first haemostasis valve clip arm 55 showing a proximal end of the dilator 54.

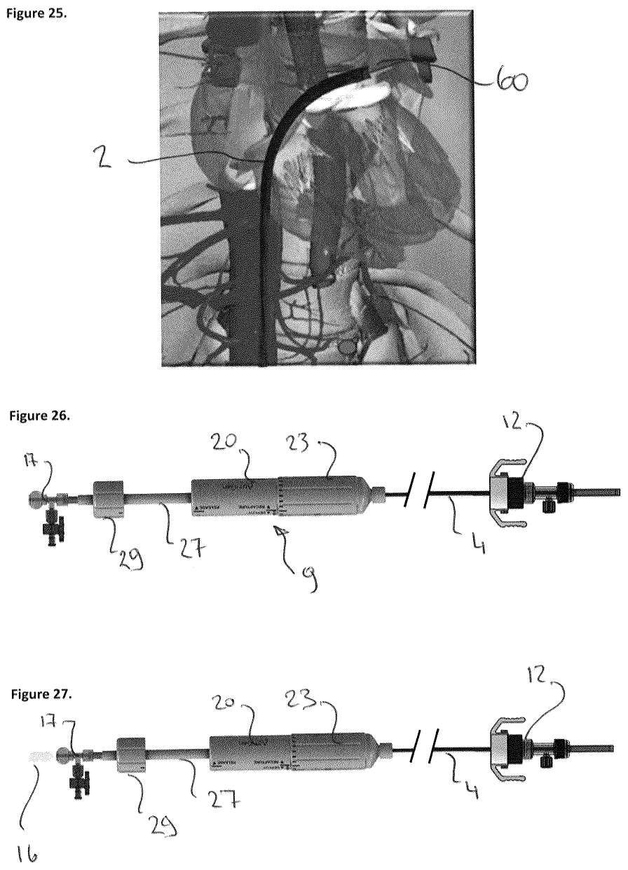

In a second step (FIG. 24), the pre-assembled access sheath/dilator is inserted into the femoral vein 57 through a conventional introducer 58, and advanced over a guidewire 59 until a distal end 13 of the access sheath 2 is positioned at a target location (in this case, the left atrium of the heart 60). The first haemostasis valve 10 prevents blood loss through the access sheath 2. FIG. 25 illustrates the access sheath 2 and dilator 54 tracked over the guidewire along the femoral artery and into the left atrium 60.

Once the access sheath 2 is positioned in the left atrium 60, the dilator 54 may be removed. At this stage, the handle 9, implant system and anchor system are assembled. FIG. 26 illustrates the pre-assembly of the handle 9, implant system (implant 6 and implant delivery shaft 4) and a loading tool comprising a second haemostasis valve 12 and haemostasis valve clip arm 61. Once assembled, the third haemostasis valve 17 at the proximal end of the handle 9 is opened, and the anchor system (anchor 8 and anchor delivery shaft 7) is advanced through the third haemostasis valve 17 and through the implant delivery shaft 4 (FIG. 27) to a position where the anchor 8 is located just proximal of the proximal hub 42 of the implant 6.

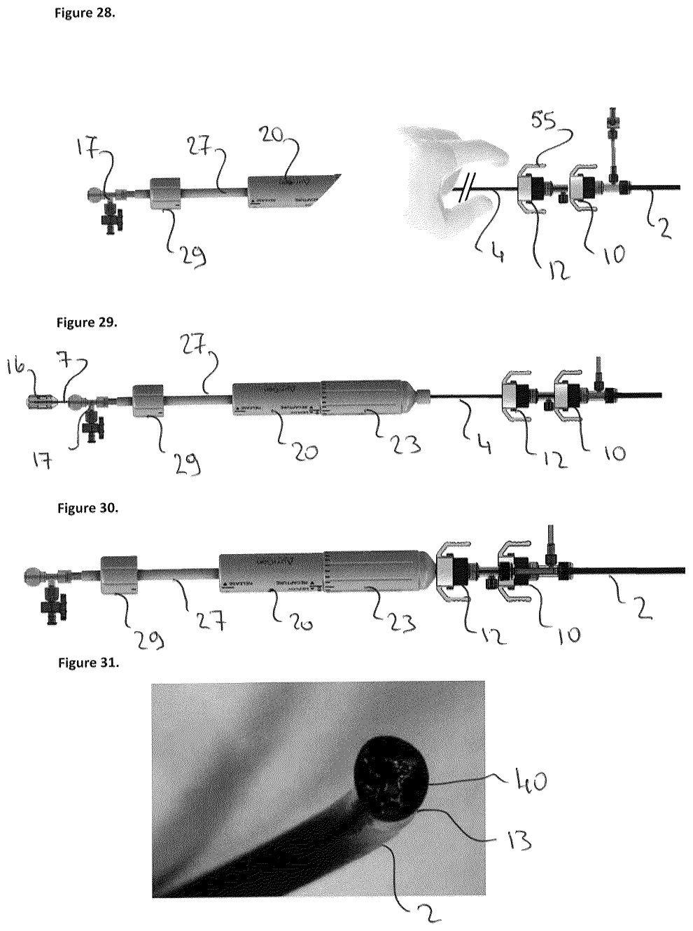

FIG. 28 illustrates the second haemostasis valve 12 being attached to the first haemostasis valve 10 while the access sheath 2 is position in the vasculature.

FIG. 29 illustrates the second haemostasis valve 12 being turned anti-clockwise to allow advancement of the implant system and anchor system through the second haemostasis valve and along the sheath 2.

FIG. 30 illustrates the second haemostasis valve 12 coupled to the handle 9 after the implant system and anchor system have been advanced through the access sheath 2 to a position where the implant is positioned just proximal of a distal end of the access sheath.

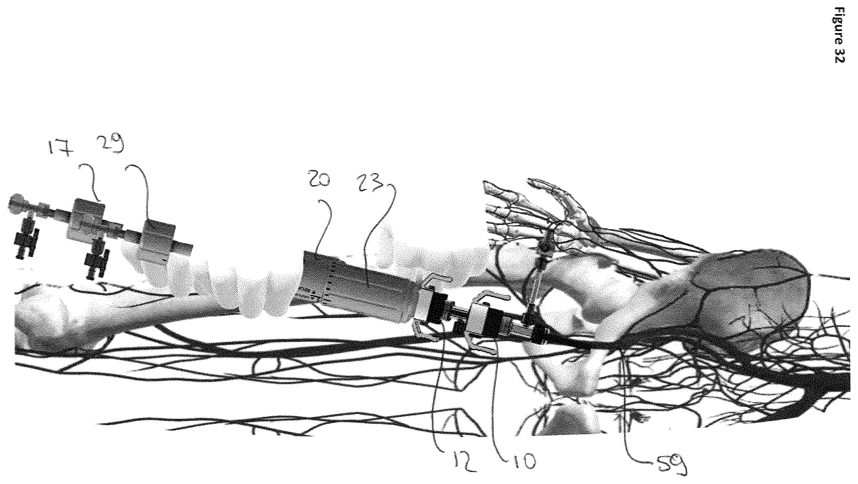

FIG. 31 illustrates the implant 6 positioned just proximal of a distal end 13 of the access sheath 2.

FIG. 32 illustrates the handle 9 and implant system (and anchor system) advanced through the access sheath.

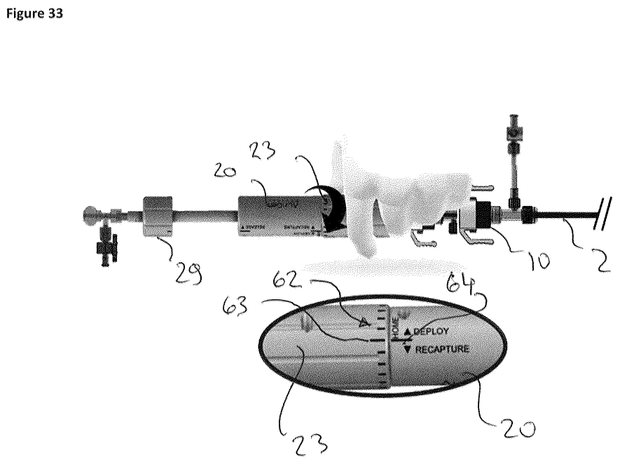

FIG. 33 illustrates the use of the actuator sleeve 23 to deploy the implant 6 from the distal end 13 of the access sheath 2.

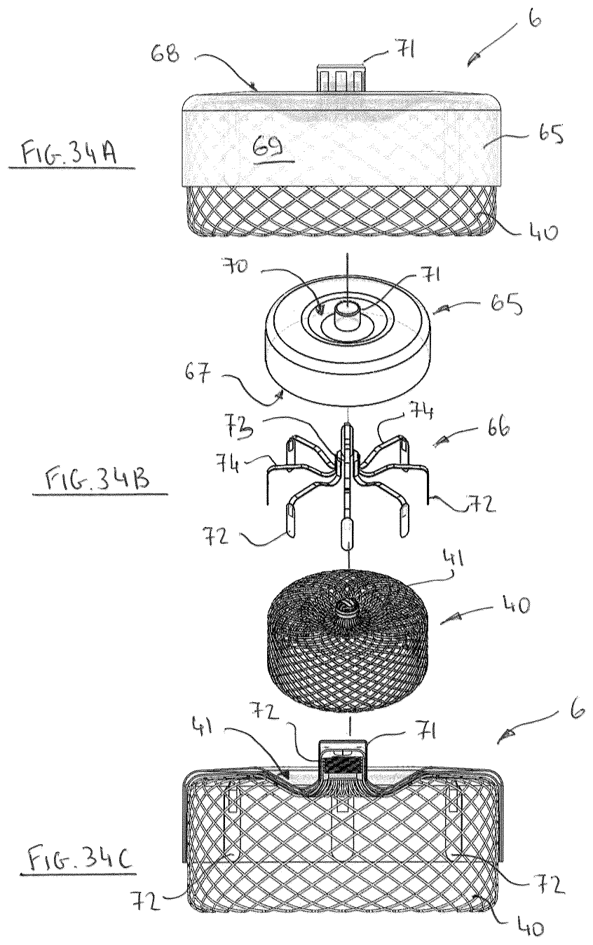

FIG. 34 illustrates the implant used in the system according to one embodiment of the invention, in which parts described with reference to previous embodiments are assigned the same reference numerals. The implant 6 is shown assembled in FIGS. 34A and 34C and the components of the implant are shown in an exploded configuration in FIG. 34B. The cover 65 has an open distal end 67 and a closed proximal end 68 and sidewall 69. The proximal end 68 has a recessed section 70 and a hub section 71 that projects proximally from the recess. The proximal end 68 of the cover is dimensioned to closely abut the proximal end 41 of the mesh cage 40. The electrode array 66 comprises eight electrodes 72 each connected to a central electrode hub 73 by a conducting wire 74. To assembly the implant, the electrode array 66 is mounted on an external surface of the mesh cage 40 and the cover 65 is then mounted over the electrode array so that the electrodes the electrodes 72 are disposed in between the mesh cage and the cover, as illustrated in the sectional view of the implant of FIG. 34C.

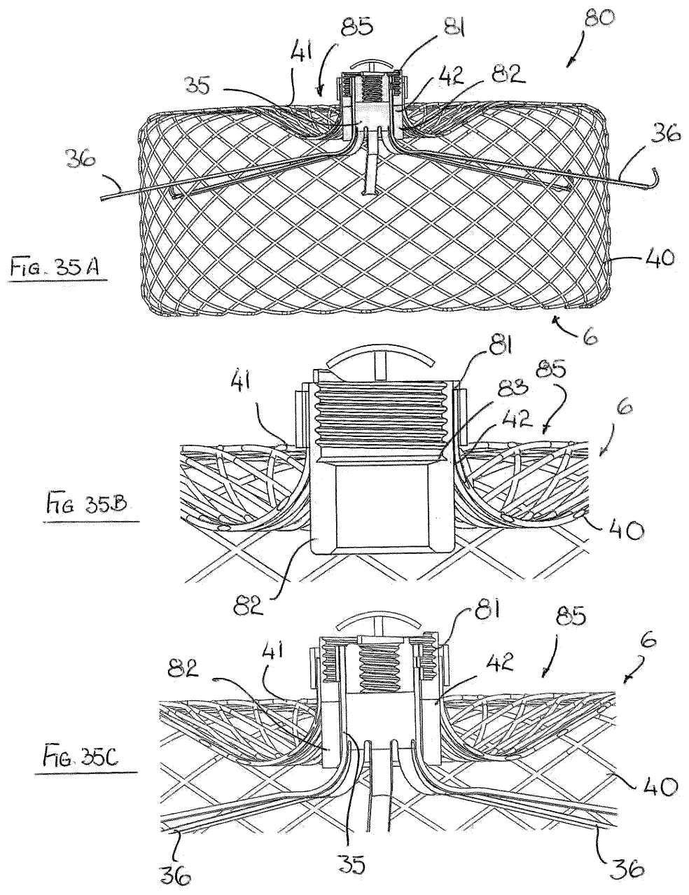

FIGS. 35A to 35C illustrate a further embodiment of the system of the invention, in which parts described with reference to the previous embodiments are assigned the same reference numerals. In this embodiment, the system, indicated generally by the reference numeral 80, comprises an implant 6 with a proximal hub 42 (recessed conduit) having an internally threaded proximal end 81, a distal end 82 and internal annular shoulder 83 disposed between the distal and proximal ends. The shoulder provides a platform against which the proximal anchor hub 35 abuts when the anchor hub 35 is nested in the proximal hub 42 of the implant. The implant comprises a mesh cage 40 having a proximal face 41 with an annular recessed section 85 surrounding the proximal hub 42. As illustrated in FIG. 35B, the distal end 82 of the proximal hub 42 extends distally of the annular recessed section 85. This ensures that when the self-deploying anchoring arms 36 are advanced through the hub 32 and pivot radially outwardly, the arms 36 are urged away from the struts of the mesh cage 40.

In an aspect on the invention, the anchor system may not be pre-loaded into the handle and implant delivery shaft and may be advanced through the handle and implant delivery shaft after the implant has been deployed and prior to the implant and implant delivery shaft being decoupled. In these embodiments, the method typically comprises advancing the handle to advance the implant delivery system (without the anchor system pre-loaded) through the access sheath. In these embodiments, the method comprises a step of treatment of tissue of the body lumen (or sensing a parameter of the tissue) after the implant has been deployed and prior to advancement and deployment of the anchor system. For example, the treatments step may comprise ablation of tissue of the body lumen, for example using a tissue energising module such as electrodes. The tissue energising module (or sensor) may be attached to the implant, or may be separate from the implant and advanced through the implant delivery sheath and deployed at the target location. Treatment may comprise a number of treatment steps, in which the implant is fully or partially re-sheathed after the first treatment step, and then re-positioned (e.g. using the handle) and a second treatment step carried out.

### REFERENCE NUMERALS

Medical implant system 1
Access sheath 2
Access sheath lumen 3
Implant delivery shaft 4
Implant delivery shaft lumen 5
Implant 6
Anchor delivery shaft 7
Anchor 8
Handle 9
First (access sheath) haemostasis valve 10
Distal coupling conduit 11
Second (implant delivery shaft) haemostasis valve 12
Distal end access sheath 13
Distal end of handle 14
Proximal section (of anchor delivery shaft) 15
Anchor shaft hub 16
Third (anchor delivery shaft) haemostasis valve 17
Main body (of handle) 20
Longitudinal axis (of handle) 21
Central bore (of handle) 22
Actuator sleeve 23
Recessed distal section (of main body) 24
Central bore (of shaft) 25
Actuator arm 26
Shaft (of actuator arm) 27
Proximal section (of actuator arm) 28
Rotary actuator 29
Proximal end (of shaft) 30
Helical groove 32
Anchor proximal hub 35
Anchoring arms 36
Mesh cage 40
Proximal end (of mesh cage) 41
Recessed hub (of mesh cage) 42
Sidewall (of mesh cage) 43
Ostium of left atrial appendage 51
Marker band 52
Wall of left atrial appendage 53
Dilator 54
First haemostasis valve clip arm 55
Proximal end of the dilator 56
Femoral vein 57
Introducer 58
Guidewire 59
Left atrium 60
Second haemostasis valve clip arm 61
Graduated markings sleeve 62
Centre marking 63
Graduated marking main body 64
Cover 65
Electrode array 66
Cover open distal end 67
Cover closed proximal end 68
Cover sidewall 69
Cover recessed proximal section 70
Cover hub section 71
Electrode 72
Electrode hub 73
Conducting wire 74
System 80
Internally threaded proximal end (proximal hub) 81
Proximal hub distal end 82
Internal annular shoulder (hub) 83
Annular recessed section (implant) 85

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1-23. (canceled)

24. A medical implant system comprising:
an access sheath having a lumen;
an implant module mountable within and axially adjustable relative to the lumen of the access sheath and comprising an implant that is radially adjustable from a contracted delivery configuration to a radially expanded deployed configuration detachably coupled to an implant delivery shaft having a lumen; and
an implant anchoring module comprising an anchor and an anchor delivery shaft detachably coupled to the anchor, wherein the anchor is radially adjustable from a contracted delivery configuration to a radially expanded implant anchoring configuration,
in which the implant anchoring module is mountable within, and axially adjustable relative to, the lumen of the implant delivery shaft.

25. The medical implant system of claim 24, in which the anchor comprises a proximal portion of an anchoring hub and a plurality of anchoring arms extending distally from a distal portion of the anchoring hub.

26. The medical implant system of claim 24, in which the anchor comprises a proximal portion of an anchoring hub and a plurality of anchoring arms extending distally from a distal portion of the anchoring hub, in which the plurality of anchoring arms are configured to self-deploy into contact with a wall of a body lumen when the plurality of anchoring arms are advanced distally through the proximal portion of the anchoring hub of the implant.

27. The medical implant system of claim 24, in which a proximal face of the implant comprises a recessed conduit and a proximal anchoring hub is configured to nest within the recessed conduit when anchoring arms are fully deployed to limit any further distal movement of the medical implant system.

28. The medical implant system of claim 24, in which a distal end of the access sheath comprises a fluorescent or radio-opaque marker.

29. The medical implant system of claim 24, in which the implant comprises a mesh cage with a proximal hub configured for detachable attachment to a distal end of the implant delivery shaft, in which the proximal hub optionally comprises a fluorescent or radio-opaque marker.

30. The medical implant system of claim 24, in which the implant comprises one or more electrodes.

31. The medical implant system of claim 24, in which the implant comprises at least one electrode, wherein the implant delivery shaft comprises an electrical supply lead configured to deliver electrical energy from an external energy source to the at least one electrode.

32. The medical implant system of claim 24, in which the implant comprises at least one sensor which is optionally a Micro Electro-Mechanical System (MEMS) sensor.

33. The medical implant system of claim 24, in which the implant anchoring module comprises at least one sensor which is optionally a Micro Electro-Mechanical System (MEMS) sensor.

34. A medical implant system (1) comprising:
an access sheath (2) having a lumen (3);
an implant system disposed within and axially adjustable relative to the lumen (3) of the access sheath (2) and comprising an implant (6) that is radially adjustable from a contracted delivery configuration to a radially expanded deployed configuration detachably coupled to an implant delivery shaft (4) having a lumen (5);
an anchoring system mounted within and axially adjustable relative to the lumen (5) of the implant delivery shaft (4) comprising an anchor (8) that is radially adjustable from a contracted delivery configuration to a radially expanded implant anchoring configuration detachably coupled to an anchor delivery shaft (7); and
a handle (9) configured to be operatively coupled to the implant delivery shaft (4) and access sheath (2) and comprising an axial actuator to adjust an axial position of the implant delivery shaft (4) relative to the access sheath (2) to deploy and/or recapture the implant (6), wherein the axial actuator comprises an external actuator sleeve (23) rotatably mounted to main body (20) of the handle (9) for rotation about a longitudinal axis (21) of the handle (9), wherein the external actuator sleeve (23) forms part of an external surface of the handle.

35. A medical implant system according to claim 34, in which the handle (9) is configured to rotate the implant delivery shaft (4) relative to the main body (20) of the handle (9) to detach the implant delivery shaft (4) from the implant (6).

36. A medical implant system according to claim 34, in which the main body (20) of the handle (9) comprises a central bore (22) disposed along the longitudinal axis (21) of the handle (9), and the axial actuator for the implant delivery shaft (4) comprises:
an actuator arm (26) disposed in the central bore (22) configured for axial and non-rotational movement relative to the central bore; and
a translation mechanism configured to convert rotational movement of the external actuator sleeve (23) to axial movement of the actuator arm (26),
wherein the implant delivery shaft (4) is attached to the actuator arm (26) for axial movement therewith.

37. A medical implant system according to claim 34, in which the anchor delivery shaft (7) extends through the handle (9) and has a proximal section (15) comprising an anchor shaft hub (16) that extends proud of a proximal end of the handle (9).

38. A medical implant system according to claim 34, comprising a first haemostasis valve (10) for the access sheath (2), a second haemostasis valve (12) for the implant delivery shaft (4), and a third haemostasis valve (17) for the anchor delivery shaft (4).

39. A medical implant system according to claim 34, wherein a working length of the implant delivery shaft (4) and a working length of the access sheath (2) are configured such that upon assembly of an implant module within the lumen (3) of the access sheath (2) and prior to deployment of the axial actuator of the handle, the implant is positioned no more than 50 mm proximally of a distal end of the access sheath.

40. A medical implant system (1) comprising:
an access sheath (2) having a lumen (3);
an implant system disposed within and axially adjustable relative to the lumen (3) of the access sheath (2) and comprising an implant (6) that is radially adjustable from a contracted delivery configuration to a radially expanded deployed configuration, and an implant delivery shaft (4) having a lumen (5);
an implant anchoring system mounted within and axially adjustable relative to the lumen (5) of the implant delivery shaft (4) comprising an anchor (8) that is radially adjustable from a contracted delivery configuration to a radially expanded implant anchoring configuration detachably coupled to an anchor delivery shaft (7); and
a handle (9) configured to be operatively coupled to the implant delivery shaft (4) and access sheath (2) and comprising an axial actuator to adjust an axial position of the implant delivery shaft (4) relative to the access sheath (2) to deploy and/or recapture the implant (6),
wherein the implant comprises a mesh cage with a proximal conduit, and wherein the implant delivery shaft (4) is detachably coupled to the proximal conduit of the implant to form an extension of the lumen (5) of the implant delivery shaft (4).

41. The medical implant system of claim 40, in which the proximal conduit forms a hub of the implant.

42. A medical implant system comprising:
an access sheath having a lumen;
an implant module mountable within and axially adjustable relative to the lumen of the access sheath and comprising an implant that is radially adjustable from a contracted delivery configuration to a radially expanded deployed configuration detachably coupled to an implant delivery shaft having a lumen; and
an implant anchoring module comprising an anchor and an anchor delivery shaft detachably coupled to the anchor, wherein the anchor is radially adjustable from a contracted delivery configuration to a radially expanded implant anchoring configuration,
in which the implant anchoring module is mountable within, and axially adjustable relative to, the lumen of the implant delivery shaft, and wherein the anchor comprises a sensor configured to sense a parameter of a left atrium.
